(19) United States
(12) Plant Patent Application Publication
(10) Pub. No.: US 2015/0096092 P1
(43) Pub. Date: Apr. 2, 2015

(54) Sedum plant named 'Touchdown Jade'

(50) ;

(71) Applicant: Terra Nova Nurseries, Inc., Canby, OR (US)

(72) Inventor: Janet N. Egger, Wilsonville, OR (US)

(73) Assignee: Terra Nova Nurseries, Inc., Canby, OR (US)

(21) Appl. No.: 13/998,103

(22) Filed: Sep. 30, 2013

(51) Int. Cl. A01H 5/00

(52) U.S. Cl. PLT/479

(57) ABSTRACT

A new and distinct hybrid of Sedum plant substantially as shown and described, with light pink flowers in large inflorescences, blue green leaves with rose red main veins, strong rose pink stems, orange carpels when the seed is developing, a multiple crown, dense, low, mounding habit, and excellent vigor.

### BOTANICAL DENOMINATION

Sedum telephium (Syn. Hylolelephium telephium)

### VARIETY DESIGNATION

‘Touchdown Jade’

### BACKGROUND OF THE INVENTION

The present invention relates to a new and distinct cultivar of Sedum telephium and given the cultivar name of ‘Touchdown Jade’. Sedum is in the family Crassulaceae. The new cultivar is part of a planned breeding program to make short, compact, multicrown plants. The new cultivar originated from an open pollinated cross of a proprietary seedling, Sedum 384-2.

Compared to the seed parent, Sedum 384-2, the new cultivar, has a better habit and larger inflorescences.

Compared to Sedum ‘Desert Blonde’, U.S. Plant patent application Ser. No. 13/815,309, the new cultivar has light pink flowers rather than creamy white.

Compared to Sedum ‘Yellow Xenox’, U.S. Plant Pat. No. 21,528, the new variety is has light pink flowers rather than cream, a wider habit, with more crowns from young plants (an advantage to a nursery grower as the plants fill the pots quickly).

This new and unique Sedum is distinguished by:

1. light pink flowers in large inflorescences,

2. blue green leaves with rose red main veins,

3. strong rose pink stems,

4. orange carpels when the seed is developing,

5. a multiple crown, dense, low, mounding habit, and

6. excellent vigor.

This new cultivar has been reproduced only by asexual propagation (division and tissue culture). Each of the progeny exhibits identical characteristics to the original plant. Asexual propagation by division and tissue culture using standard micropropagation techniques with terminal and lateral shoots, as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWINGS

FIG. 1 shows an 18 month old plant of Sedum ‘Touchdown Jade’ in the garden in late May in Canby, Oreg.

FIG. 2 shows a 10 month old plant in flower and fruit in the trial field in late August in Canby, Oreg.

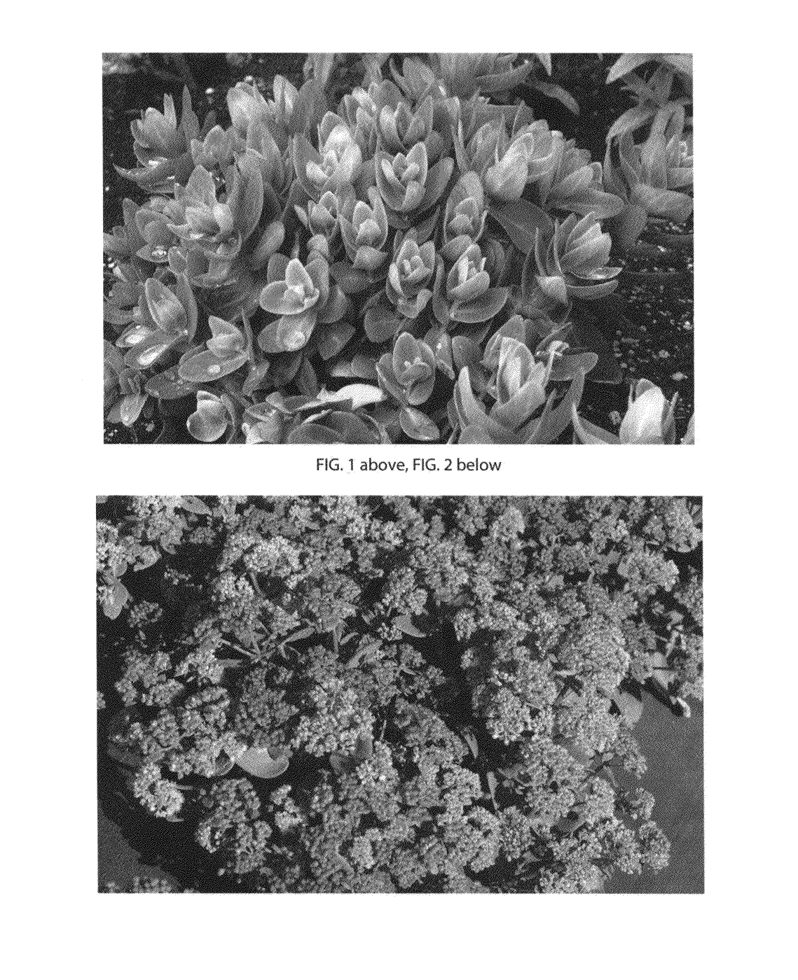

### DETAILED PLANT DESCRIPTION

The following is a detailed description of the new Sedum hybrid based on observations of 10 month old specimens grown in the trial bed in full sun under typical outdoor conditions in Canby, Oreg. Canby is Zone 8 on the USDA Hardiness map. Temperatures range from a high of 95° F. in August to 32° F. in January. Normal rainfall in Canby is 42.8 inches per year. The color descriptions are all based on The Royal Horticultural Society Colour Chart, 5^{th} edition.
Plant:
   Type.—herbaceous perennial.
   Hardiness.—USDA Zone 4 to 9.
   Size.—22 cm tall from the ground to the base of the inflorescence and 48 cm wide.
   Habit.—low, clumping mound.
   Vigor.—excellent.
Stem:
   Number.—about 8.
   Size.—13 cm long and 6 mm wide.
   Internode length.—4 cm to 5 cm long.
   Color.—closest to Yellow Green 147C.
   Texture.—succulent.
   Surface.—glabrous.
Leaf:
   Type.—simple.
   Arrangement.—opposite.
   Shape.—ovate.
   Apex.—obtuse.
   Base.—obtuse.
   Venation.—pinnate.
   Margins.—entire to slightly serrate, tinted Red Purple 187B.
   Blade size.—grows to 11 cm long and 5.8 cm wide.
   Surface.—glabrous on both sides.
   Texture.—thick, spongy, succulent.
   Petiole.—leaves sessile.
   Leaf color.—topside Green 137B with the vein 147D; bottom side Green 138B with vein 147D.
Inflorescence:
   Type.—terminal, corymbose, umbel-like.
   Number of flowers.—about 500.
   Pedicels.—2 mm to 3 mm long, 1 mm wide, glabrous, Yellow Green 148B.
   Rachis—grows to 8 cm tall and 6 mm wide, Greyed Purple 184B in sun, Yellow Green 147D where shaded, glabrous.
   Size.—grows to 10 cm deep and 12 cm wide.
   Bloom period.—August through September in Canby, Oreg.
Flower bud:
   Size.—2.5 mm wide, 4 mm deep.
   Description.—ovoid.
   Color.—Greyed Red 182C.
Flower:
   Type.—actinomorphic.
   Shape.—stellate.
   Size.—5 mm deep and 5 mm wide.
   Corolla description.—5 mm deep and 5 mm wide, 5 petals, each lanceolate, 4.5 mm long and 1.5 mm wide, margins entire, tip acuminate, glabrous on both sides, topside Red Purple 65C and bottom side Red Purple 65C on top half blending to White N155C.
   Calyx description.—1.5 mm deep and 3 mm wide, stellate, 5 lobes, divided to the base, margins entire, tip acute, glabrous, Yellow Green 148B on both sides.
   Stamen description.—10, 2.5 mm long, filaments 2.4 mm long and White NN155C, anthers sterile and reduced Grey Brown N199A, no pollen.
   Pistil description.—5 separate prominent carpels, each 4 mm deep and 1.5 mm wide, ovary 2 mm long, White NN155C except top where Red Purple 61A, style 2 mm long, stigma and style Red Purple 61A.
   Fragrance.—none.
   Lastingness.—each stem lasts up to a month.
Fruit:
   Type.—Ventrally dehiscent follicle, erect, attenuate.
   Fertility.—low.
   Size.—3 mm wide and 4 mm deep.
   Color.—Greyed Orange N170A.
Seed: None seen
Disease and pest resistance or tolerance: Pest and disease resistance are typical for this type of Sedum. No known resistances.

I claim:

1. A new and distinct hybrid of Sedum plant as herein illustrated and described.
